(19) United States
(12) Patent Application Publication
(10) Pub. No.: US 2026/0207464 A1
(43) Pub. Date: Jul. 23, 2026

(54) COSMETIC COMPOSITION COMPRISING NIACINAMIDE, A GINGER ROOT EXTRACT, A CARBOXYLIC ACRYLIC POLYMER, AN ACTIVE AGENT FOR COMBATING HAIR LOSS AND/OR AN ANTI-DANDRUFF ACTIVE AGENT

(71) Applicant: L'OREAL, Paris (FR)

(72) Inventors: Christophe BOULLE, Saint Ouen (FR); Olivia TOURIGUINE, Saint Ouen (FR); Mathilde RIVIERE, Saint Ouen (FR)

(21) Appl. No.: 19/139,215

(22) PCT Filed: Dec. 13, 2023
(86) PCT No.: PCT/EP2023/085487

§ 371 Date: Jun. 13, 2025

(30) Foreign Application Priority Data
Dec. 16, 2022 (FR) FR2213542

(51) Int. Cl. A61K 8/81; A61K 8/34; A61K 8/49; A61K 8/67; A61K 8/92; A61K 8/9794; A61Q 5/00; A61Q 7/00

(52) U.S. Cl. CPC A61K 8/8152; A61K 8/345; A61K 8/4926; A61K 8/4953; A61K 8/675; A61K 8/922; A61K 8/9794; A61Q 5/006; A61Q 7/00; A61K 2800/54; A61K 2800/805

(57) ABSTRACT

Cosmetic composition comprising niacinamide, a ginger root extract, a carboxylic acrylic polymer, an active agent for combating hair loss and/or an anti-dandruff active agent

The present invention relates to a cosmetic composition comprising niacinamide, a ginger root extract, at least one carboxylic acrylic polymer, at least one active agent for combating hair loss and/or at least one anti-dandruff active agent.

The invention also relates to a process for the cosmetic treatment of human keratin materials such as the skin, notably human scalp, hair and/or eyelashes, comprising at least the application of the cosmetic composition according to the present invention.

The present invention also relates to the use of said cosmetic composition for treating the loss and/or promoting the regrowth of keratin fibres.

The present invention also relates to the use of said cosmetic composition for cosmetically treating dandruff.

The present invention relates to a cosmetic composition comprising niacinamide, a ginger root extract, at least one carboxylic acrylic polymer, at least one active agent for combating hair loss and/or at least one anti-dandruff active agent.

The invention also relates to a process for the cosmetic treatment of human keratin materials such as the skin, notably human scalp, hair and/or eyelashes, comprising at least the application of the cosmetic composition according to the present invention.

The present invention also relates to the use of said cosmetic composition for treating the loss and/or promoting the regrowth of keratin fibres.

The present invention also relates to the use of said cosmetic composition for cosmetically treating dandruff.

The present invention relates to the field of the cosmetic treatment of human keratin materials, in particular human keratin fibres, such as human head hair, eyebrows, eyelashes and/or bodily hair, notably beard hair, moustache hair and pubic hair, and of the skin, notably the scalp and areas of the skin, in particular alopecic or non-alopecic areas, preferably alopecic areas, covered with keratin fibres as mentioned previously. More particularly, the invention relates to the cosmetic treatment of human hair and/or eyelashes and/or skin, preferably hair and/or scalp.

The growth and renewal of the hair in humans are mainly determined by the activity of the hair follicles and of their dermo-epidermal environment. Their activity is cyclical and essentially comprises three phases, namely the anagenic phase, the catagenic phase and the telogenic phase.

The anagenic phase (corresponding to the active or growth phase), which lasts several years and during which the hairs lengthen, is followed by a very short and transient catagenic phase which lasts a few weeks. During this phase, the hair strand undergoes a change, the follicle atrophies and its dermal implantation appears higher and higher.

The terminal phase, which lasts a few months, corresponds to a resting phase known as the telogenic phase. At the end of this resting period, the hair strands fall out and are replaced by new hair follicles in the anagenic phase, in such a way that another hair cycle begins.

Head hair is thus constantly being renewed and, of the approximately 150 000 hair strands which make up a head of hair, at any one moment approximately 10% of them are at rest and will thus be replaced in the months to come.

Alopecia denotes accelerated loss of head hair, eyelashes, eyebrows and/or bodily hair in any part of the body. It generally occurs in genetically predisposed individuals and mainly affects men. This is known as androgenetic or androgenic alopecia, or even androgeno-genetic alopecia.

Alopecia is essentially due to a disturbance in hair renewal, which initially leads to an acceleration in the frequency of the cycles at the expense of the hair quality and then of its quantity. This results in a gradual impoverishment of the hair by the regression of the “terminal” hairs to the downy stage. Certain areas are preferentially affected: notably in men, the temporal or frontal lobes and also the upper part of the occipital lobe, whereas in women diffuse alopecia of the vertex is seen.

Other causes may bring about substantial temporary or permanent hair loss. These may include hair loss and impairments after pregnancy (or post partum), during malnutrition or dietary imbalances, or even asthenia or hormonal dysfunction, as may be the case during or after the menopause. It may also be a case of hair loss or impairments related to seasonal phenomena.

Alopecia may also affect the eyelashes, eyebrows and/or bodily hair on any part of the human body, and may cause aesthetic displeasure in the same way as alopecia affecting one or more areas of the scalp.

People who are thus prone to alopecia are increasingly resorting to cosmetic treatments that enable them to eliminate and/or reduce the effect of the alopecia, and notably to reduce or delay the loss of head hair, eyelashes and/or bodily hair, and also to induce or stimulate their growth.

Such treatments generally consist of daily applications to the various alopecic areas of the human body, in particular to alopecic areas of the scalp, such as the temporal or frontal lobes in men, or areas of the body free of hair, in particular on the face, notably the beard, moustache and/or eyebrows, of cosmetic compositions comprising one or more agents for combating hair loss and/or for promoting hair regrowth.

To this end, agents such as minoxidil, finasteride, aminexil or stemoxydine may notably be mentioned as agents that are capable of reducing the loss and/or promoting the regrowth of keratin fibres.

The duration of these treatments generally ranges from several weeks to several months, and may even extend over years, and their long-term efficacy most often depends on assiduous regular application by the user of the cosmetic compositions to the areas to be treated, which may be more or less extensive.

The application of treatments that are capable of reducing the loss and/or promoting the regrowth of head hair, eyelashes and/or bodily hair may notably last several weeks before the desired effects can begin to be seen, in particular curbing alopecia.

These treatments must be used daily to obtain pronounced efficacy.

However, these compositions still too often have numerous application drawbacks which may have a negative impact on the cosmetic treatment to be followed, notably leading possibly to more or less rapid abandonment of the treatment and, consequently, to poor results in terms of limiting or even eliminating alopecia.

Specifically, many compositions include gelling agents in order to prevent the composition from running onto the face, into the eyes or down the nape of the neck. However, the use of gelling agents, notably in the context of daily use, leads to aggregation of the keratin fibres, giving the head of hair a tacky effect that is unacceptable to consumers.

In order to improve the efficacy of these treatments, it may be necessary to increase the amounts of active agents, which may lead to difficulties in dissolving the active agents and to the formation of powders that are visible on the keratin fibres and scalp during use. This powdery effect is not acceptable to consumers either. Lotions for combating hair loss must be used daily to obtain pronounced efficacy. Some consumers wash their hair every day, in which case the products previously applied to the scalp are eliminated. Most consumers do not wash their hair every day, and so the product applied on the second day is added to the product applied on the first day. This is known as a cumulative effect. In this context of repeated application without washing the scalp, the appearance of powder may be present from the first day, and usually on the second day. This appearance of powder results from the precipitation of the solid compounds in the formula when the solvent evaporates.

As a result, the insufficient application qualities of these compositions may in the long term discourage users from assiduously following cosmetic treatments for combating hair loss and/or promoting hair regrowth, all the more so as the efficacy of these treatments is mainly linked to the regular daily application of the compositions described previously to the areas to be treated for several weeks or even several months.

In other words, a lack of regularity or abandonment of the treatment too quickly usually leads to poor results in terms of efficacy in combating hair loss and/or promoting hair regrowth and, consequently, to dissatisfaction. This further deepens the disappointment among consumers, who do not have the time to observe the long-term progress and/or efficacy of these treatments.

There is thus a real need to overcome the drawbacks mentioned previously, notably by using a composition that is capable of cosmetically and effectively treating the loss of human keratin fibres such as human head hair, bodily hair and/or eyelashes, and/or of promoting their regrowth, which has improved application qualities while at the same time notably affording a satisfactory visual result, notably without a greasy or tacky effect and without residue. This composition must also allow repeated application, i.e. allow daily use of the composition.

This aim is achieved by the present invention, one subject of which is notably a cosmetic composition comprising:
i) niacinamide,
ii) a ginger root extract,
iii) one or more carboxylic acrylic polymers,
iv) one or more active agents for combating hair loss and/or one or more anti-dandruff active agents,
v) optionally one or more hydroxylated compounds with a melting point below 25° C. at atmospheric pressure (1.013×10⁵ Pa) (e.g. they are liquid at 25° C. and atmospheric pressure) and a boiling point of greater than or equal to 170° C., preferably chosen from hydroxylated compounds consisting of a C2-C8 hydrocarbon-based chain optionally interrupted with an oxygen atom and comprising from one to two free hydroxyl groups (—OH) borne by different carbon atoms.

The cosmetic composition according to the invention allows a gelled texture to be obtained, thus preventing running, but without producing a greasy or tacky effect on the fibres.

The cosmetic composition of the invention does not cause the product to transfer onto the fingers during application or during the day. The fingers are thus not greasy or tacky when the composition is used.

The cosmetic composition according to the invention also has improved application qualities by avoiding the powdery effect and/or aggregation on keratin fibres, in particular in the context of repeated application required for frequent use.

The cosmetic composition according to the invention is also entirely satisfactory in terms of its efficacy in combating the loss and/or promoting the regrowth of keratin fibres and/or in combating dandruff. It also has a beneficial effect on increasing the diameter of the keratin fibres and on strengthening the fibres against breakage.

In addition, the composition according to the invention gives good cosmetic properties, notably conditioning properties, to keratin fibres, particularly in terms of sheen, suppleness, disentanglement and a smooth feel.

The invention also relates to a process for the cosmetic treatment of human keratin materials such as the skin, notably human scalp, hair and/or eyelashes, comprising at least the application of the cosmetic composition according to the present invention.

The present invention also relates to the use of said cosmetic composition for treating the loss and/or promoting the regrowth of keratin fibres and/or for cosmetically treating dandruff.

The term “dandruff treatment” means the cosmetic treatment of dandruff, notably to delay or prevent its appearance.

The present invention also relates to the use of said cosmetic composition for increasing the diameter of keratin fibres and/or for reinforcing keratin fibres against breakage.

Other subjects, characteristics, aspects and advantages of the invention will emerge even more clearly on reading the description and the example that follows.

In the text hereinbelow, unless otherwise indicated, the limits of a range of values are included in that range, notably in the expressions “between” and “ranging from . . . to . . . ”.

Moreover, the expression “at least one” used in the present description is equivalent to the expression “one or more”.

### i) Niacinamide

The cosmetic composition according to the invention comprises niacinamide (3-pyridinecarboxamide).

Niacinamide is also known as nicotinamide.

Preferably, the total content of niacinamide ranges from 1% to 5% by weight, preferentially from 1.5% to 4.5% by weight and better still from 2% to 4% by weight relative to the total weight of the composition.

### ii) Ginger Root Extract

The cosmetic composition according to the invention also comprises a ginger root extract (INCI name: Zingiber officinale root extract).

The ginger root extract may be in crushed, powdered or liquid form, preferably in liquid form.

The ginger root extract may notably be obtained by supercritical CO₂ extraction from ginger root fragments, for example ginger root powder.

The total content of ginger root extract preferably ranges from 0.02% to 0.5% by weight, more preferentially from 0.03% to 0.3% by weight, better still from 0.05% to 0.2% by weight relative to the total weight of the composition.

iii) Carboxylic Acrylic Polymers

The cosmetic composition according to the invention also comprises one or more carboxylic acrylic polymers.

The carboxylic acrylic polymer(s) may be chosen from associative and non-associative polymers and mixtures thereof, preferably from associative polymers. Thus, preferably, the cosmetic composition according to the invention also comprises one or more associative carboxylic acrylic polymers.

For the purposes of the present invention, the term “acrylic polymer” means a polymer comprising at least one unit derived from an α,β-monoethylenically unsaturated carboxylic acid monomer, derivatives thereof and/or salts thereof, preferably chosen from acrylic acid, methacrylic acid, derivatives thereof and/or salts thereof.

It is recalled that “associative polymers” are polymers that are capable, in an aqueous medium, of reversibly associating with each other or with other molecules.

Their chemical structure more particularly comprises at least one hydrophilic zone and at least one hydrophobic zone.

The term “hydrophobic group” means a radical or polymer with a saturated or unsaturated, linear or branched hydrocarbon-based chain, comprising at least 10 carbon atoms, preferably from 10 to 30 carbon atoms, in particular from 12 to 30 carbon atoms and more preferentially from 18 to 30 carbon atoms.

Preferentially, the hydrocarbon-based group originates from a monofunctional compound. By way of example, the hydrophobic group may be derived from a fatty alcohol such as stearyl alcohol, dodecyl alcohol, decyl alcohol or behenyl alcohol. It may also denote a hydrocarbon-based polymer, for instance polybutadiene.

Among the associative carboxylic acrylic polymers that may be used in the present invention, mention may notably be made of:
(a) those including at least one hydrophilic unit and at least one fatty-chain allyl ether unit, more particularly those whose hydrophilic unit is constituted by an ethylenically unsaturated anionic monomer, even more particularly by a vinylcarboxylic acid and most particularly by an acrylic acid or a methacrylic acid or mixtures thereof.

Among these polymers, the ones that are particularly preferred according to the invention are polymers formed from 20% to 60% by weight of acrylic acid and/or of methacrylic acid, from 5% to 60% by weight of lower alkyl (meth)acrylates, from 2% to 50% by weight of fatty-chain allyl ether, and from 0 to 1% by weight of a crosslinking agent which is a well-known copolymerizable unsaturated polyethylenic monomer, for instance diallyl phthalate, allyl (meth)acrylate, divinylbenzene, (poly)ethylene glycol dimethacrylate or methylenebisacrylamide.

Among the latter polymers, the ones most particularly preferred are crosslinked terpolymers of methacrylic acid, of ethyl acrylate and of polyethylene glycol (10 EO) stearyl alcohol ether (Steareth-10), notably those sold by the company Ciba under the names Salcare SC 80® and Salcare SC 90®, which are aqueous 30% emulsions of a crosslinked terpolymer of methacrylic acid, of ethyl acrylate and of steareth-10 allyl ether (40/50/10);
(b) those including i) at least one hydrophilic unit of unsaturated olefinic carboxylic acid type, and ii) at least one hydrophobic unit of the type such as a (C₁₀-C₃₀)alkyl ester of an unsaturated carboxylic acid.

(C₁₀-C₃₀)alkyl esters of unsaturated carboxylic acids that are useful in the invention comprise, for example, lauryl acrylate, stearyl acrylate, decyl acrylate, isodecyl acrylate, dodecyl acrylate and behenyl acrylate, and the corresponding methacrylates, lauryl methacrylate, stearyl methacrylate, decyl methacrylate, isodecyl methacrylate, dodecyl methacrylate and behenyl methacrylate.

Polymers of this type are described and prepared, for example, according to patents U.S. Pat. Nos. 3,915,921 and 4,509,949.

Among the polymers of this type that will be used more particularly are those consisting of from 60% to 95% by weight of acrylic acid (hydrophilic unit), 4% to 40% by weight of C₁₀-C₃₀ alkyl acrylate (hydrophobic unit) and 0 to 6% by weight of crosslinking polymerizable monomer, or alternatively those consisting of from 96% to 98% by weight of acrylic acid (hydrophilic unit), 1% to 4% by weight of C₁₀-C₃₀ alkyl acrylate (hydrophobic unit) and 0.1% to 0.6% by weight of crosslinking polymerizable monomer such as those described previously.

Among said polymers above, the ones most particularly preferred according to the present invention are the products sold by the company Goodrich under the trade names Pemulen TR1®, Pemulen TR2®, Carbopol 1382®, and even more preferentially Pemulen TR1®, and the product sold by the company SEPPIC under the name Coatex SX®.

Mention may also be made of the acrylic acid/lauryl methacrylate/vinylpyrrolidone terpolymer sold under the name Acrylidone LM by the company ISP.
(c) acrylic terpolymers comprising:
i) approximately 20% to 70% by weight of an α,β-monoethylenically unsaturated carboxylic acid [A],
ii) approximately 20% to 80% by weight of an α,β-monoethylenically unsaturated non-surfactant monomer other than [A],
iii) approximately 0.5% to 60% by weight of a nonionic monourethane which is the reaction product of a monohydric surfactant with a monoethylenically unsaturated monoisocyanate,
such as those described in patent application EP-A-0 173 109 and more particularly the terpolymer described in Example 3, namely a methacrylic acid/methyl acrylate/behenyl alcohol dimethyl meta-isopropenylbenzylisocyanate ethoxylated (40 EO) terpolymer, as an aqueous 25% dispersion.
(d) copolymers including, among their monomers, an α,β-monoethylenically unsaturated carboxylic acid and an ester of an α,β-monoethylenically unsaturated carboxylic acid and of an oxyalkylenated fatty alcohol.

Preferentially, these compounds also comprise, as monomer, an ester of an α,β-monoethylenically unsaturated carboxylic acid and of a C₁-C₄ alcohol.

An example of a compound of this type that may be mentioned is Aculyn 22® sold by the company Röhm & Haas, which is a methacrylic acid/ethyl acrylate/oxyalkylenated stearyl methacrylate terpolymer; and also Aculyn 88, also sold by the company Röhm & Haas. Mention may also be made of Novethix L-10 Polymer sold by the company Lubrizol, having the INCI name Acrylates/Beheneth-25 Methacrylate Copolymer.

Preferably, the carboxylic acrylic polymer(s) are chosen from associative carboxylic acrylic polymers, more preferentially from copolymers including among their monomers an α,β-monoethylenically unsaturated carboxylic acid and an ester of an α,β-monoethylenically unsaturated carboxylic acid and of an oxyalkylenated fatty alcohol, notably an ester of an α,β-monoethylenically unsaturated carboxylic acid and of an oxyalkylenated, notably oxyethylenated, C₁₀-C₂₄ and preferably C₁₅-C₂₄ alcohol, in particular those also comprising, as monomer, an ester of an α,β-monoethylenically unsaturated carboxylic acid and of a C₁-C₄ alcohol; even more preferentially, the carboxylic acrylic polymer is Acrylates/Beheneth-25 Methacrylate Copolymer.

Preferably, the total content of the carboxylic acrylic polymer(s) ranges from 0.05% to 4% by weight, more preferentially from 0.1% to 2% by weight, even more preferentially from 0.2% to 1% by weight, and better still from 0.3% to 0.8% by weight, relative to the total weight of the composition.

Preferably, the total content of the associative carboxylic acrylic polymer(s) ranges from 0.05% to 4% by weight, more preferentially from 0.1% to 2% by weight, even more preferentially from 0.2% to 1% by weight, and better still from 0.3% to 0.8% by weight, relative to the total weight of the composition.

### iv) Active Agents for Combating Hair Loss and Anti-Dandruff Active Agents

The cosmetic composition according to the invention also comprises one or more active agents for combating hair loss and/or one or more anti-dandruff active agents. Said active agents for combating hair loss and anti-dandruff active agents iv) are other than the compounds described previously.

Preferably, the cosmetic composition according to the invention comprises one or more active agents for combating hair loss.

Preferably, the cosmetic composition according to the invention comprises one or more anti-dandruff active agents.

Particularly preferably, the cosmetic composition according to the invention comprises one or more active agents for combating hair loss and one or more anti-dandruff active agents.

The term “agent for combating hair loss” means any compound which promotes the growth of keratin fibres and/or increases their density, and/or limits their loss.

Preferably, the agent for combating hair loss is an agent for combating the loss of human keratin fibres, such as human head hair, bodily hair and/or eyelashes.

Preferably, the agent for combating hair loss is an agent for combating the loss of human hair and/or eyelashes, and more preferentially hair.

Thus the agent is more preferentially an agent for promoting hair growth and/or increasing its density, and/or limiting its loss.

The active agent(s) for combating hair loss may be chosen from the following compounds, and also mixtures thereof:
(A) 2,4-diaminopyrimidine 3-N-oxide and/or an organic or mineral acid salt thereof and/or a hydrate thereof;
2,4-Diaminopyrimidine 3-N-oxide (INCI name: Diaminopyrimidine oxide) has the following formula:

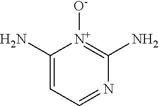

Among the organic or mineral acid salts that may be used, mention may be made in particular of the hydrochlorides, sulfates, citrates and acetates. As hydrate, mention may be made in particular of the monohydrate.

Preferably, the non-salified compound in monohydrate form is used.

(B) 2,4-diamino-6-piperidinopyrimidine 3-N-oxide and/or an organic or mineral acid salt thereof and/or a hydrate thereof.

2,4-Diamino-6-piperidinopyrimidine 3-N-oxide (or Minoxidil) corresponds to the following formula:

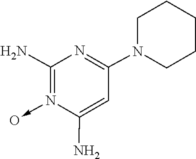

(C) pyridinedicarboxylic acid esters and/or salts thereof;

The pyridinedicarboxylic acid esters correspond to formula (I) below:

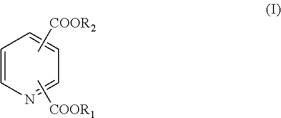

in which R₁ and R₂ represent, independently of each other, a hydrogen atom, a linear or branched, saturated or unsaturated (notably alkyl) C1-C18 aliphatic hydrocarbon-based radical; or a C6-C18 aryl radical; said aliphatic hydrocarbon-based radicals or aryl radicals being optionally substituted with one or more groups chosen from OH, NH₂, —OR, —OCOR and —NHR with R representing a C1-C18 alkyl group; it being understood that at least one of the groups R₁ and R₂ is other than a hydrogen atom.

Preferably, the substituents COOR₁ and COOR₂ are in positions 2 and 3 or in positions 2 and 4, respectively, of the pyridine ring. Preferentially, they are in positions 2 and 4.

Preferentially, the compounds correspond to formula (Ia):

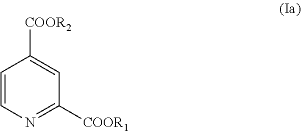

in which R₁ and R₂ represent, independently of each other, a hydrogen atom, a linear or branched, saturated or unsaturated (notably alkyl) C1-C18 aliphatic hydrocarbon-based radical; or a C6-C18 aryl radical; said aliphatic hydrocarbon-based or aryl radicals being optionally substituted with one or more groups chosen from OH, NH₂, —OR, —OCOR and —NHR with R representing a C1-C18 alkyl group, at least one of the groups R₁ and R₂ being other than a hydrogen atom.

Preferably, in formulae (I) and (Ia), the aliphatic hydrocarbon-based radical, notably a C1-C18 alkyl radical, is an aliphatic hydrocarbon-based radical, notably a C1-C10 alkyl radical, notably a C1-C6 alkyl radical, such as methyl, ethyl, tert-butyl, isopropyl or hexyl. Said aliphatic hydrocarbon-based radical may also contain at least one carbon-carbon double bond or one carbon-carbon triple bond, for instance —CH═CH₂, —CH2-CH═CH—CH₃ or —CH₂—CCH.

The aryl radical may represent the phenyl or naphthyl radical.

In particular, in formulae (I) and (Ia), R₁ and R₂ represent, independently of each other, a hydrogen atom, a saturated aliphatic hydrocarbon-based radical, notably a linear or branched C1-C18, better still C1-C10, or even C1-C6 alkyl radical, optionally substituted with an alkoxy or acyloxy group (OR or OCOR with R denoting a C1-C18 alkyl radical), at least one of the groups R₁ and R₂ being other than a hydrogen atom.

Preferably, R₁ and R₂ are identical.

Preferentially, R₁ and R₂ are identical and represent a saturated and linear C1-C18, preferably C1-C10, and better still C1-C6, alkyl radical, and most particularly an ethyl radical.

Mention may notably be made of the compounds of formula (I) below:
dimethyl pyridine-2,4-dicarboxylate,
dimethyl pyridine-2,3-dicarboxylate,
diethyl pyridine-2,4-dicarboxylate,
diethyl pyridine-2,3-dicarboxylate,
diethyl pyridine-2,5-dicarboxylate,
dimethyl pyridine-2,5-dicarboxylate,
diisopropyl pyridine-2,4-dicarboxylate,
di(acetyloxymethyl)pyridine-2,4-dicarboxylate (derivative of formula (I) such that R₁ and R₂ represent -CH2-O—COCH3).

According to the invention, the term “salts of the compounds of formula (I)” means the organic or mineral salts of a compound of formula (I), these salts being physiologically acceptable. As mineral salts, mention may be made of sodium or potassium salts and also the zinc (Zn²⁺), calcium (Ca²⁺), copper (Cu²⁺), iron (Fe²⁺), strontium (Sr²⁺), magnesium (Mg²⁺) or manganese (Mn²⁺) salts; hydroxides, carbonates and chlorides. As organic salts, mention may be made of the triethanolamine, monoethanolamine, diethanolamine, hexadecylamine, N,N,N′,N′-tetrakis(2-hydroxypropyl)ethylenediamine and tris(hydroxymethyl)aminomethane salts.

(D) O-acylated derivatives obtained by partial or total esterification of vitamin F with glucose, nicotinic acid esters, menthol and derivatives thereof, notably menthoxypropanediol, vitamins such as vitamin D, vitamin B12 analogues, panthenol, vitamin B8 and biotin (vitamin H), may also be mentioned as active agents for combating hair loss that can be used.

Preferably, active agent(s) for combating hair loss are chosen from 2,4-diaminopyrimidine 3-N-oxide and/or an organic or mineral acid salt thereof and/or a hydrate thereof; more preferentially, the active agent for combating hair loss is 2,4-diaminopyrimidine 3-N-oxide in monohydrate form.

When it is (they are) present in the cosmetic composition according to the invention, the total content of active agents for preventing hair loss iv) preferably ranges from 0.05% to 8% by weight, more preferentially from 0.1% to 5%, even more preferentially from 0.2 to 4%, from 0.5% to 3%, and better still from 1% to 2% by weight, relative to the total weight of the composition.

When it is present in the cosmetic composition according to the invention, the total content of 2,4-diaminopyrimidine 3-N-oxide and/or an organic or mineral acid salt thereof and/or a hydrate thereof preferably ranges from 0.05% to 8% by weight, better still from 0.1% to 5%, even better still from 0.2 to 4%, better still from 0.5% to 3%, and preferentially from 1% to 2% by weight, relative to the total weight of the composition.

When it is present in the cosmetic composition according to the present invention, the content of 2,4-diaminopyrimidine 3-N-oxide and/or a hydrate thereof, notably a monohydrate, ranges from 0.05% to 8% by weight, better still from 0.1% to 5%, even better still from 0.2% to 4%, from 0.5% to 3% and preferentially from 1% to 2% by weight, relative to the total weight of the composition.

The term “anti-dandruff active agent” means any compound which prevents or limits excessive, visible desquamation of the scalp.

The anti-dandruff active agent(s) may be chosen, alone or as mixtures, from the following compounds:
(A) 1-hydroxy-2-pyridone derivatives:

The 1-hydroxy-2-pyridone derivatives are preferably chosen from the compounds of formula (A1) or salts thereof:

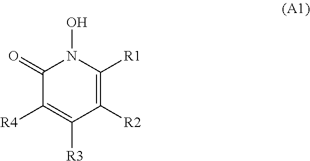

in which:
   R1 denotes a hydrogen atom; a linear or branched alkyl group containing from 1 to 17 carbon atoms, notably C6-C12; a cycloalkyl group containing 5 to 8 carbon atoms; a cycloalkyl-alkyl group, the cycloalkyl group containing 5 to 8 carbon atoms and the alkyl group containing from 1 to 4 carbon atoms; an aryl or aralkyl group, the aryl group containing from 6 to 30 carbon atoms and the alkyl group containing from 1 to 4 carbon atoms; an aryl-alkenyl group, the aryl group containing from 6 to 30 carbon atoms and the alkenyl group containing from 2 to 4 carbon atoms; the cycloalkyl and aryl groups as defined above may be substituted with one or more alkyl groups containing 1 to 4 carbon atoms or else one or more alkoxy groups containing from 1 to 4 carbon atoms;
   R2 denotes a hydrogen atom; an alkyl group containing from 1 to 4 carbon atoms; an alkenyl group containing from 2 to 4 carbon atoms; a halogen atom or a benzyl group;
   R3 denotes a hydrogen atom, an alkyl group containing from 1 to 4 carbon atoms or a phenyl group; and
   R4 denotes a hydrogen atom; an alkyl group containing from 1 to 4 carbon atoms; an alkenyl group containing from 2 to 4 carbon atoms; a methoxymethyl group; a halogen atom or a benzyl group.

Preferably, R2=R4=H.

Preferably, R3=methyl.

Preferably, R1 is a C6-C12 branched alkyl radical.

Among these compounds, 1-hydroxy-4-methyl-6-(2,4,4-trimethylpentyl)-2-(1H)-pyridone and 6-cyclohexyl-1-hydroxy-4-methyl-2-(1H)-pyridone are preferred.

Among the salts that may be used, mention may be made of the salts of lower (C1-C4)alkanolamines, such as ethanolamine and diethanolamine, amine or alkylamine salts, and also salts with inorganic cations, for instance ammonium salts and the salts of alkali metals or alkaline-earth metals.

The monoethanolamine salt of 1-hydroxy-4-methyl-6-(2,4,4-trimethylpentyl)-2 (1H)-pyridinone (more commonly known as piroctone olamine or octopirox) is most particularly preferred.
(B) Ellagic acid and derivatives thereof:

Ellagic acid, or 2,3,7,8-tetrahydroxy-(1)-benzopyrano (5,4,3-cde)(1) benzopyran-5,10-dione, is a well-known molecule present in the plant kingdom. Reference may be made to the publication in the Merck Index, 20th edition (1996), No. 3588.

Ellagic acid has the following chemical formula:

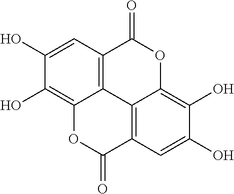 [SMILES: O=c1oc2c(O)c(O)cc3c(=O)oc4c(O)c(O)cc1c4c23]

which includes four fused rings.

The ellagic acid ether(s) that may be used according to the invention are preferably chosen from the mono-, di-, tri- or polyethers obtained by etherification of one or more hydroxyl groups (one of the four OH groups of ellagic acid) of ellagic acid to one or more groups OR, R being chosen from C2-C20 alkyl groups, polyoxyalkylene groups, and in particular polyoxyethylene and/or polyoxypropylene groups, and groups derived from one or more mono- or polysaccharides, for instance the group having the following formula:

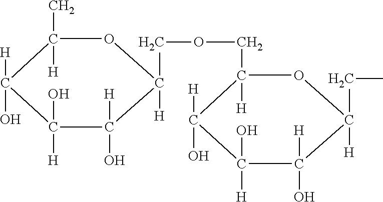

In the case of the di-, tri- or polyethers of ellagic acid, the groups R as defined above may be identical or different.

Preferably, these ellagic acid ethers are chosen from 3,4-di-O-methylellagic acid, 3,3′-4-tri-O-methylellagic acid and 3,3′-di-O-methylellagic acid.

The salt(s) of ellagic acid and/or the ethers thereof that may be used according to the invention are preferably chosen from the alkali metal or alkaline-earth metal salts, such as the sodium, potassium, calcium and magnesium salt, the ammonium salt and the salts of amines such as the triethanolamine, monoethanolamine, arginine and lysine salts. Preferably, the salt(s) of ellagic acid and/or the ethers thereof that may be used according to the invention are chosen from the salts of alkali metals or alkaline-earth metals, notably the sodium, potassium, calcium or magnesium salts.
(C) pyrithione and derivatives thereof:

Pyrithione is the compound 1-hydroxy-2 (1H)-pyridinethione or 2-pyridinethiol 1-oxide.

The pyrithione salts that may be used in the context of the invention are notably monovalent metal salts and divalent metal salts such as the sodium, calcium, magnesium, barium, strontium, zinc, cadmium, tin and zirconium salts. The divalent metal salts and in particular the zinc salt (zinc pyrithione) are particularly preferred.
(D) selenium (poly) sulfides, among which mention may be made of selenium disulfide and selenium polysulfides of formula SeₓS_{y} in which x and y are numbers between 1 and 7, and such that x+y=8. Selenium disulfide is in the form of a powder, the particles of which generally have a particle size of less than 200 μm, preferably less than 25 μm.

Preferably, the anti-dandruff active agent is chosen from the 1-hydroxy-2-pyridone derivatives of formula (A1) or salts thereof; more preferentially, the anti-dandruff active agent is the monoethanolamine salt of 1-hydroxy-4-methyl-6-(2,4,4-trimethylpentyl)-2(1H)-pyridinone (more commonly known as piroctone olamine or octopirox).

Preferably, the total content of anti-dandruff active agent(s) iv) ranges from 0.05% to 2% by weight, more preferentially from 0.1% to 1%, even more preferentially from 0.1% to 0.5% by weight, relative to the total weight of the composition.

Preferably, the total content of piroctone olamine ranges from 0.05% to 2% by weight, more preferentially from 0.1% to 1%, even more preferentially from 0.1% to 0.5% by weight, relative to the total weight of the composition.

Preferably, the cosmetic composition according to the invention also comprises one or more active agents for combating hair loss, chosen from 2,4-diaminopyrimidine 3-N-oxide and/or an organic or mineral acid salt thereof and/or a hydrate thereof.

Preferably, the cosmetic composition according to the invention also comprises piroctone olamine as an anti-dandruff active agent.

Particularly preferably, the cosmetic composition according to the invention also comprises one or more anti-dandruff active agents chosen from 2,4-diaminopyrimidine 3-N-oxide and/or an organic or mineral acid salt thereof and/or a hydrate thereof and piroctone olamine as anti-dandruff active agent.

### v) Hydroxylated Compounds

The cosmetic composition according to the invention may optionally also comprise one or more hydroxylated compounds v) with a melting point below 25° C. at atmospheric pressure (1.013×10⁵ Pa) (e.g. they are liquid at 25° C. and atmospheric pressure) and a boiling point of greater than or equal to 170° C.

Preferably, said hydroxylated compounds v) consist of a C2-C8 hydrocarbon-based chain optionally interrupted with an oxygen atom and comprising from one to two free hydroxyl groups (—OH) borne by different carbon atoms.

These compounds may be cyclic or acyclic, linear or branched, saturated or unsaturated.

Preferably, the hydroxylated compound(s) v) are chosen from linear C2-C8 and better still C2-C6 diols or aromatic C6-C8 and better still C7-C8 monoalcohols and mixtures thereof. More preferentially, the hydroxylated compound(s) v) are chosen from linear C2-C8 and better still C2-C6, diols.

Mention may be made in particular of propylene glycol, butylene glycol, pentylene glycol, hexylene glycol, 1,3-propanediol, benzyl alcohol, phenethyl alcohol (phenylethyl alcohol) and dipropylene glycol, and even more preferentially the hydroxylated compound v) is propylene glycol.

Preferably, when they are present in the composition according to the invention, the total content of hydroxylated compound(s) v) is greater than or equal to 1% by weight, more preferentially greater than or equal to 1.5% by weight, relative to the total weight of the composition.

Preferably, when they are present in the composition according to the invention, the total content of hydroxylated compound(s) v) ranges from 1% to 15% by weight, better still from 1.5% to 10% by weight, more preferentially from 2% to 8% by weight, even more preferentially from 2.1% to 6% by weight, better still from 2.2% to 5% by weight, even better still from 2.3% to 4% by weight, relative to the total weight of the composition.

Preferably, when they are present in the composition according to the invention, the total content of hydroxylated compounds v) chosen from linear C2-C8 diols is greater than or equal to 1% by weight, better still ranges from 1% to 15% by weight, preferentially ranges from 1.5% to 10% by weight, more preferentially from 2% to 8% by weight, even more preferentially from 2.1% to 6% by weight, better still from 2.2% to 5% by weight, even better still from 2.3% to 4% by weight, relative to the total weight of the composition.

Preferably, when it is present in the composition according to the invention, the total content of propylene glycol is greater than or equal to 1% by weight, better still ranges from 1% to 15% by weight, preferentially from 1.5% to 10% by weight, more preferentially from 2% to 8% by weight, even more preferentially from 2.1% to 6% by weight, better still from 2.2% to 5% by weight, even better still from 2.3% to 4% by weight, relative to the total weight of the composition.

### vi) Solvents

The composition according to the invention may optionally comprise one or more organic solvents other than the hydroxylated compounds v) described previously.

These organic solvents are preferably chosen from C1-C5 monoalcohols, preferentially C2-C4 monoalcohols. Particularly preferably, ethanol is used.

Preferably, the composition comprises one or more organic solvents other than the hydroxylated compounds v) described previously, preferentially one or more C2-C4 monoalcohols, better still ethanol.

When it is (they are) present, the total content of organic solvents other than the hydroxylated compounds v) preferably ranges from 1% to 60% by weight, preferentially from 5% to 50% by weight, more preferentially from 10% to 40% by weight, better still from 20% to 35% by weight, relative to the total weight of the composition.

When the composition comprises ethanol, the ethanol content preferably ranges from 1% to 60% by weight, preferentially from 5% to 50% by weight, more preferentially from 10% to 40% by weight, better still from 20% to 35% by weight, relative to the total weight of the composition.

The composition is preferably aqueous, the water content possibly ranging from 10% to 90% by weight, preferably from 20% to 80% by weight and preferentially from 40% to 70% by weight relative to the total weight of the composition.

### Additives

The composition may optionally also comprise one or more additives other than the compounds described previously, such as those chosen from cationic, anionic, amphoteric or zwitterionic, nonionic surfactants and mixtures thereof, cationic, anionic, nonionic, amphoteric polymers or mixtures thereof, anti-seborrhoeic agents, vitamins and pro-vitamins including panthenol, sunscreens, sequestrants, plasticizers, solubilizing agents, acidifying agents, opacifying or nacreous agents, antioxidants, hydroxy acids, fragrances, preserving agents and ceramides.

Needless to say, a person skilled in the art will take care to select this or these optional additional compounds such that the advantageous properties intrinsically associated with the composition according to the invention are not, or are not substantially, adversely affected by the envisaged addition(s).

The above additives may generally be present in an amount, for each of them, of between 0 and 20% by weight relative to the total weight of the composition.

### Process

The invention also relates to a process for the cosmetic treatment of human keratin materials such as the skin, notably human scalp, hair and/or eyelashes, comprising at least the application of the cosmetic composition as defined previously.

The composition according to the present invention is preferably applied topically, notably to the scalp and/or the hair.

The application of the composition may or may not be followed by rinsing after a possible leave-on time.

Preferably, the application of the composition according to the invention is not followed by rinsing.

Preferably, the cosmetic treatment process is a process for caring for the hair and/or the scalp.

A subject of the present invention is also the use of a cosmetic composition as defined previously for treating the loss and/or promoting the regrowth of keratin fibres and/or for cosmetically treating dandruff.

A subject of the present invention is also the use of a cosmetic composition as defined previously for increasing the diameter of keratin fibres and/or reinforcing keratin fibres against breakage.

The examples that follow serve to illustrate the invention without, however, being limiting in nature.

### EXAMPLES

In the examples that follow, all the amounts are given as mass percentages of active material (AM) relative to the total weight of the composition (unless otherwise mentioned).

### Example 1

Composition A according to the invention was prepared using the ingredients whose contents are indicated in the tables below (in % AM):

TABLE 1

| | Composition A |
|---|---|
| | invention |
| Acrylates/Beheneth-25 | 0.45 |
| Methacrylate | |
| copolymer | |
| Propylene glycol | 2.5 |
| PEG-40 castor oil | 0.1 |
| Ethanol | 27 |
| Piroctone olamine | 0.25 |
| Diaminopyrimidine | 1.5 |
| oxide | |
| Ginger root extract | 0.1 |
| Niacinamide | 3 |
| Fragrance | 0.4 |
| Water | qs 100 |

### Example 2

### a) Compositions

Composition B (according to the invention) and composition B′ (comparative) were prepared using the ingredients whose contents are indicated in the tables below:

TABLE 2

| | Composition B | Composition B′ |
|---|---|---|
| | (invention) | (comparative) |
| Acrylates/Beheneth- | 0.45 | — |
| 25 Methacrylate | | |
| copolymer | | |
| Ammonium | — | 0.45 |
| polyacryloyldimethyl | | |
| taurate | | |
| Propylene glycol | 2.5 | 2.5 |
| PEG-40 castor oil | 0.3 | 0.3 |
| Ethanol | 28 | 28 |
| Piroctone olamine | 0.25 | 0.25 |
| Diaminopyrimidine | 1.5 | 1.5 |
| oxide | | |
| Ginger root extract | 0.1 | 0.1 |
| Niacinamide | 3 | 3 |
| Fragrance | 0.4 | 0.4 |
| Water | qs 100 | qs 100 |

### b) Protocol

5.4 g (27 cm) locks of natural Caucasian hair (HT4 Type 2) were first washed with a DOP shampoo (1.5 g of shampoo per lock).

The locks were then wet 10 times under the tap between two fingers and then squeezed dry twice between two fingers before being combed with a large-toothed comb (twice) and then a small-toothed comb (twice).

Compositions B (according to the invention) and B′ (comparative) were then applied by hand, at a rate of 0.4 g per 5.4 g lock, and spread over the entire lock. The locks were then combed with a small-toothed comb (once) and smoothed out by hand before being blow-dried with air at room temperature.

### c) Results

Three experts evaluated, under blind conditions, the visual greasiness and the tacky feel of the treated locks, and also the deposit on the fingers (product transfer):
visual greasiness: the fibres are visually shiny and stick together,
tacky feel: the fibres are stuck together,
deposit on the fingers: when the fingers are passed through the locks, there is residue on the fingers.

The locks treated with composition B′ (comparative) are visually greasier and tackier than the locks treated with composition B according to the invention.

In addition, the locks treated with composition B′ (comparative) transfer more product onto the fingers than those treated with composition B according to the invention.

Thus, the composition according to the invention affords locks which are less greasy and less tacky and which transfer less product onto the fingers.

1-15. (canceled)

16. A cosmetic composition comprising:
i) niacinamide,
ii) a ginger root extract,
iii) one or more carboxylic acrylic polymers,
iv) one or more active agents for combating hair loss,
iv′) optionally one or more anti-dandruff active agents,
v) optionally one or more hydroxylated compounds with a melting point below 25° C. at atmospheric pressure of 1.013×10⁵ Pa, and a boiling point of greater than or equal to 170° C.

17. The composition of claim 16, wherein the total amount of i) niacinamide ranges from 1% to 5% by weight, relative to the total weight of the composition.

18. The composition of claim 16, wherein the total amount of ii) ginger root extract ranges from 0.02% to 0.5% by weight, relative to the total weight of the composition.

19. The composition of claim 16, wherein the iii) one or more carboxylic acrylic polymers is chosen from associative carboxylic acrylic polymers.

20. The composition of claim 16, wherein the iii) one or more carboxylic acrylic polymers is chosen from copolymers comprising an α,β-monoethylenically unsaturated carboxylic acid and an ester of an α,β-monoethylenically unsaturated carboxylic acid and an oxyalkylenated fatty alcohol.

21. The composition of claim 16, wherein the iii) one or more carboxylic acrylic polymers is chosen copolymers comprising an ester of an α,β-monoethylenically unsaturated carboxylic acid and an oxyalkylenated C₁₀-C₂₄ alcohol.

22. The composition of claim 16, wherein the total amount of iii) carboxylic acrylic polymers ranges from 0.05% to 4% by weight, relative to the total weight of the composition.

23. The composition of claim 16, wherein the iv) one or more active agents for combating hair loss is chosen from 2,4-diaminopyrimidine 3-N-oxide, an organic or mineral acid salt thereof, and/or a hydrate thereof; 2,4-diamino-6-piperidinopyrimidine 3-N-oxide, an organic or mineral acid salt thereof, and/or a hydrate thereof; pyridinedicarboxylic acid esters and/or salts thereof; O-acylated derivatives from partial or total esterification of vitamin F with glucose; nicotinic acid esters; menthol and derivatives thereof; or mixtures thereof.

24. The composition of claim 16, wherein the total amount of iv) active agents for combating hair loss ranges from 0.05% to 8% by weight, relative to the total weight of the composition.

25. The composition of claim 16, further comprising iv′) one or more anti-dandrugg active agents, wherein the iv′) one or more anti-dandruff active agents is chosen from 1-hydroxy-2-pyridone derivatives, ellagic acid and derivatives thereof, pyrithione and derivatives thereof, selenium (poly) sulfides, or mixtures thereof.

26. The composition of claim 16, wherein the iv′) one or more anti-dandruff active agents is chosen from 1-hydroxy-2-pyridone derivatives, selenium (poly) sulfides, or mixtures thereof.

27. The composition of claim 16, wherein the iv′) one or more anti-dandruff active agents is chosen from 1-hydroxy-2-pyridone derivatives of formula (A1) or salts thereof: 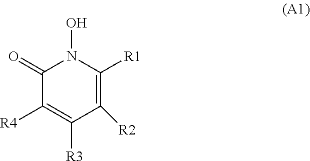
wherein:
R1 denotes a hydrogen atom; a linear or branched alkyl group comprising from 1 to 17 carbon atoms; a cycloalkyl-alkyl group, the cycloalkyl group comprising 5 to 8 carbon atoms; a cycloalkyl-alkyl group, wherein the cycloalkyl group comprises 5 to 8 carbon atoms and the alkyl group comprises from 1 to 4 carbon atoms; an aryl or aralkyl group, wherein the aryl group comprises from 6 to 30 carbon atoms and the alkyl group comprises from 1 to 4 carbon atoms; an aryl-alkenyl group, wherein the aryl group comprises from 6 to 30 carbon atoms and the alkenyl group comprises from 2 to 4 carbon atoms; wherein the cycloalkyl and aryl groups as defined above may be substituted with one or more alkyl groups comprising 1 to 4 carbon atoms or may be substituted with one or more alkoxy groups comprising from 1 to 4 carbon atoms;
R2 denotes a hydrogen atom; an alkyl group comprising from 1 to 4 carbon atoms; an alkenyl group comprising from 2 to 4 carbon atoms; a halogen atom or a benzyl group;
R3 denotes a hydrogen atom, an alkyl group comprising from 1 to 4 carbon atoms or a phenyl group; and
R4 denotes a hydrogen atom; an alkyl group comprising from 1 to 4 carbon atoms; an alkenyl group comprising from 2 to 4 carbon atoms; a methoxymethyl group; a halogen atom or a benzyl group.

28. The composition of claim 16, wherein the iv′) one or more anti-dandruff active agents is a monoethanolamine salt of 1-hydroxy-4-methyl-6-(2,4,4-trimethylpentyl)-2 (1H)-pyridinone.

29. The composition of claim 16, wherein the total amount of iv′) anti-dandruff active agents ranges from 0.05% to 2% by weight, relative to the total weight of the composition.

30. The composition of claim 16, wherein the v) one or more hydroxylated compounds is chosen from linear C2-C8 diols, aromatic C6-C8 monoalcohols, or mixtures thereof.

31. The composition of claim 16, wherein the v) one or more hydroxylated compounds is chosen from propylene glycol, butylene glycol, pentylene glycol, hexylene glycol, 1,3-propanediol, benzyl alcohol, phenethyl alcohol, dipropylene glycol, or mixtures thereof.

32. The composition of claim 16, wherein the total amount of hydroxylated compounds v) is greater than or equal to 1% by weight, relative to the total weight of the composition.

33. The composition of claim 16, further comprising: vi) one or more organic solvents other than the compounds v), wherein the vi) one or more organic solvents is chosen from C1-C5 monoalcohols.

34. A method for treating human keratin materials, comprising applying to the keratin materials a composition comprising:
i) niacinamide,
ii) a ginger root extract,
iii) one or more carboxylic acrylic polymers,
iv) one or more active agents for combating hair loss,
iv′) optionally one or more anti-dandruff active agents,
v) optionally one or more hydroxylated compounds with a melting point below 25° C. at atmospheric pressure of 1.013×10⁵ Pa, and a boiling point of greater than or equal to 170° C.

35. A method for treating the loss and/or promoting the regrowth of keratin fibers and/or for cosmetically treating dandruff, comprising applying to the keratin fibers a composition comprising:
i) niacinamide,
ii) a ginger root extract,
iii) one or more carboxylic acrylic polymers,
iv) one or more active agents for combating hair loss
iv′) optionally one or more anti-dandruff active agents,
v) optionally one or more hydroxylated compounds with a melting point below 25° C. at atmospheric pressure of 1.013×10⁵ Pa, and a boiling point of greater than or equal to 170° C.
